(19) United States
(12) Patent Application Publication
(10) Pub. No.: US 2018/0311144 A1
(43) Pub. Date: Nov. 1, 2018

(54) COSMETIC COMPOSITION HAVING PROBIOTIC BACTERIA

(71) Applicants: NATURA COSMÉTICOS S.A., São Paulo (BR); UNIVERSIDADE FEDERAL DE MINAS GERAIS, Belo Horizonte (BR)

(72) Inventors: Luciana DE MIRANDA CHAVES VASQUEZ PINTO, Säo Paulo (BR); Débora DOMENES PALMIERI RODRIGUEZ, São Paulo (BR); Jacques Robert NICOLI, Belo Horizonte (BR); Flaviano DOS SANTOS MARTINS, Belo Horizonte (BR); Anna Karolina SOARES SILVA, Belo Horizonte (BR); Simone Helena DA SILVA, Belo Horizonte (BR)

(21) Appl. No.: 15/768,292

(22) PCT Filed: Oct. 17, 2016
(86) PCT No.: PCT/BR2016/050260

§ 371 Date: Apr. 13, 2018

Related U.S. Application Data
(60) Provisional application No. 62/242,017, filed on Oct. 15, 2015.

(51) Int. Cl. A61K 8/99; A61Q 19/08; A61Q 19/10

(52) U.S. Cl. CPC A61K 8/99; A61Q 19/10; A61Q 19/08

(57) ABSTRACT

The present invention relates to a probiotic cosmetic formulation for the cosmetic treatment of the skin, comprising a microbiota and a cosmetically acceptable carrier. Preferably, betaines and/or prebiotic agents may be incorporated into said formulation. The invention also relates to the use of a microbiota in preparing said probiotic cosmetic formulation of the invention, and a method for the cosmetic treatment of the skin, which comprises administering to the skin of an individual the probiotic cosmetic formulation of the invention.

### FIELD OF INVENTION

The present invention relates to cosmetic compositions that may be present in soap bars, emulsions, gels, creams or lotions comprising probiotic ingredients and cosmetically acceptable excipients.

### PRIOR ART

The human skin defines the boundary between the external and the internal body environment, functioning as the first line of defense against external pathogens. But it also contains several symbiont microorganisms such as bacteria, fungi and viruses, and the organization of such microorganisms is called microbiota.

Particularly on the skin, there are bacteria important for maintaining the firmness, resistance and natural mechanisms of skin defense, fundamentally contributing to good skin health maintenance. Staphylococcus epidermidis is the main bacteria of the skin, for it synthesizes protease, keratinase, lipases and nucleases that contribute to the physiological renewal mechanisms of the skin even during aging.

In addition, the skin comprises harmful bacteria such as Propionibacterium acnes and Staphylococcus aureus, which are responsible for a number of skin disorders. Probiotics are microorganisms that interact with the microbiota present in the skin, and while stimulating the production of its defenses and strengthening the internal architecture of the skin, they can also provide an increased production of lumican and periostin. Prebiotics are ingredients that favor the beneficial bacteria and protect the skin from harmful bacteria.

Lumican and periostin are glycosaminoglycans (GAG's) which maintain the collagen fiber structure. Lumican is a keratan sulfate which inhibits spontaneous lateral growth of collagen fibers, keeping the longitudinal direction. The presence of lumican ensures strength and tensile strength to the collagen structure. Periostin is a GAG that modulates the communication between cells and the extracellular matrix regulating the physiological tissue remodeling and wound healing processes. The expression of periostin and lumican is modulated by pro- and anti-inflammatory cytokines.

Antibacterial cosmetic compositions existing in prior art affect both the beneficial bacteria and harmful bacteria present on the skin, and the excessive use of said compositions weakens the natural defenses of the skin, leaving it more vulnerable to pathogens.

Said antibacterial cosmetic compositions may have toxicity to the user and the environment and do not prevent the recolonization. Examples of antibacterial ingredients are triclosan, triclocarban, benzalkonium chloride and aluminum chlorohydrate.

Therefore, there remains the need to cosmetic compositions having antibacterial selectivity, i.e. able to control the harmful bacteria, without affecting the beneficial bacteria present on the skin.

### DESCRIPTION OF THE INVENTION

Therefore it is an objective of this invention to provide a cosmetic composition having antibacterial selectivity comprising probiotic ingredients capable of treating and maintaining the firmness, resistance and natural mechanisms of renewal and defense of the skin.

According to the meaning of the present invention, the “antibacterial selectivity” means that specific probiotics included in the cosmetic compositions will inhibit the proliferation of certain bacteria and will have a less effective inhibition upon others; in this case the selectivity desired may be towards S. epidermis.

This selective effect is important to regulate the microbiota's overall metabolism, in particular the production of lactic acid, which helps to maintain an acidic pH of the skin. The microbiota also produces antimicrobial peptides (AMP's) that protect the skin from pathogens, which are often associated with pathologies such as dermatitis, rosacea and psoriasis. The microbiota is also associated with the regulation of vitamin D production and the production of extracellular matrix proteins such as hyaluronic acid and enzymes important to maintaining the skin barrier such as sphingomyelinase.

A particular embodiment of the present invention is a cosmetic composition comprising probiotics and cosmetically acceptable excipients.

Another embodiment of the present invention concerns the use of said probiotics as a selective antibacterial ingredient in order to prepare a cosmetic composition.

Still another embodiment of the present invention concerns a cosmetic method of simultaneous cleaning and treatment of the skin comprising the application of said cosmetic composition upon the skin of a user.

The present invention provides probiotics that work in novel biological mechanisms having an antibacterial selectivity while providing increased production of collagen and lumican. These biological mechanisms confer firmness, strengthening, and recover the architecture of the skin, while also reducing excessive sensitivity. Moreover, the selective antibacterial activity helps to reduce skin contamination without damaging its balance.

The probiotics of the present invention are selected from the group of bifidobacteria. Particularly, the bifidobacteria according to the present invention stimulate the skin to produce substances which reduce the response to external stimuli and prevent recolonization by Propionibacterium acnes and Staphylococcus aureus, without unbalancing the skin microbiota, being selective towards Staphylococcus epidermidis, preventing skin aging and protecting it from environmental aggressions. The bifidobacteria used in the present invention also stimulate the production of collagen and lumican, increasing the firmness of the dermis and enhancing its structure. The bifidobacteria according to the present invention are selected from the strain Bifidobacteria longum 5^{1A}; Bifidobacteria bifidum 162^{2A}; Bifidobacteria breve 110^{1A}; Bifidobacteria pseudolongum 119^{1A}.

In a preferred embodiment such bacteria are used in a concentration from about 10⁵ to about 10³ cells/ml.

In a particular embodiment, the probiotics used in the present invention may be combined with natural components that have prebiotic activity. These natural components may be selected from one or more vegetable oils and/or butters selected from the group comprising acai oil, andiroba oil, burity oil, passionflower oil, palm olein, cupuacu butter, cocoa butter, murumuru, patauá oil, ucuhuba seed oil, or a mixtures thereof.

Thus, the present invention provides a cosmetic composition comprising probiotics having unexpected simultaneous antibacterial selectivity and antiaging effect, while also preventing recolonization from exogenous and potentially harmful microorganisms.

Cosmetically acceptable excipients according to the present invention are known in the art, for instance raw materials cited in The International Cosmetic Ingredient Dictionary and Handbook (INCI).

In a particular embodiment, the cosmetic composition may further comprise sunscreens, antioxidants and sensory modifiers.

Sunscreens, without limiting the scope of the present invention may be selected among bemotrizinola (bis etilhexiloxifenol methoxyphenyl triazine), dimethylamino hidroxibenzoila hexyl benzoate, ethylhexyl methoxycinnamate, Homosalate, bisoctrizola (Tinosorb M), ethylhexyl triazone, or mixtures thereof.

Antioxidants, without limiting the scope of the present invention may be selected among butylated hydroxytoluene (BHT), tocopherol acetate or natural plant extracts, for example, Camellia sinensis (green tea), Theobroma cacao (cocoa), or mixtures thereof.

Sensory modifiers, without limiting the scope of the present invention may be selected among silicones, such as dimethicone or cyclopentasiloxane, or between other compounds such as isopropyl titanium triisostearate, crospolymers chosen from cyclopentasiloxane/dimethicone, nylon-12, polymethylsilsesquioxane or mixtures thereof.

The present invention may be included in various cosmetic products for the face and body, makeup, deodorants, oral and personal hygiene, dandruff control and seborrhea, diaper rash creams, post-treatment products such as post-peeling and post-shaving, creams, gels, lotions, ointments and soaps.

The following examples, without imposing any limitation, illustrate the present invention, which surprisingly provides an antibacterial selectivity as well as an antiaging effect.

### EXAMPLES

### Example 1. Evaluation In Vitro of the Antiaging Effect

Bifidobacteria were tested both active and inactivated in conditioned medium templates to assess whether they were able to indirectly stimulate fibroblasts. The model consists of conditioned medium in the incubation of keratinocytes with the probiotics, the removal of the supernatant and this one further incubated with fibroblasts. The cytokines profiles were measured in the supernatants of both cells and matrix proteins were measured in the supernatant of the fibroblasts.

The following tables show the results of the tests, indicating whether there was an increase or decrease in the profiles of the cytokines.

TABLE 1
Summary for IL-8, IL-6 and IL-10

| | | IL-8 | IL-6 | IL-10 |
|---|---|---|---|---|
| B bifidum | active | decrease | increase | increase |
| | inactive | decrease | increase | increase |
| B. breve | active | decrease | increase | increase |
| | inactive | decrease | increase | |
| B. longum | active | decrease | increase | increase |
| | inactive | decrease | increase | increase |
| B. pseudolongum | active | decrease | increase | |
| | inactive | decrease | | |

TABLE 2
Summary for collagen, periostin and lumican

| | | Collagen | Periostin | Lumican |
|---|---|---|---|---|
| B. bifidum | active | increase | increase | increase |
| | inactive | increase | | increase |
| B. breve | active | | increase | |
| | inactive | increase | | increase |
| B. longum | active | | increase | |
| | inactive | increase | | increase |
| B. pseudolongum | active | increase | | increase |
| | inactive | increase | | increase |

Tables 1 and 2 summarize the cytokine profile for both active and inactivated bifidobacteria. The reduction in IL-8 indicates that the probiotic of the present invention does not stimulate response from the inflammatory pathways to external agents. The IL-6 is increase in vivo means an increase of Th2 lymphocyte recruitment, which is responsible for the standard inflammatory response to pathogens. The IL-10 increase is also important for the recruitment of a specific subpopulation of Th2 lymphocytes, regulatory T cells (Treg), these cells acting on the fine adjustment of the intensity of the inflammatory response.

Overall this balance of cytokines allows inferring that the probiotic of the present invention signals the skin that there are bacteria on its surface, but not pathogenic, reducing excessive sensitivity of the skin.

It can also be interpreted that all the inactivated and some active bifidobacteria were able to stimulate increased synthesis of collagen by fibroblasts, the performance of inactivated bifidobacteria being surprising, surpassing the effects of active bacteria. The same expression profile was observed for lumican.

### Example 2. Antibacterial Selectivity

The antimicrobial activity was tested in vitro with a variation of the inhibition halo model, where the live bifidobacteria were plated and then inactivated before the inoculation with microorganisms present in the microbiota of the human skin: Propionibacterium acnes (ATCC 6916 and ATCC 51277); Staphylococcus aureus (ATCC 29213 and ATCC 25923); and Staphylococcus epidermidis (ATCC 12228).

TABLE 3
Antagonist activity of bifidobacteria

| | | | B. | |
|---|---|---|---|---|
| | B. longum | B. breve | pseudolongum | B. bifidum |
| | 5 1A | 110 1A | 119 1A | 162 2A |
| P. acnes | 23.05 mm | 21.99 mm | 14.77 mm | 19.3 mm |
| atcc6919 | | | | |
| P. acnes | 21.41 mm | 28.1 mm | | 32.3 mm |
| 51277 | | | | |
| S. aureus | 49.45 mm | 41.9 mm | 30.69 mm | 44.27 mm |
| 25923 | | | | |
| S. aureus | 37.05 mm | 31.13 mm | 29.98 mm | 21.68 mm |
| 29213 | | | | |
| S. epidermidis | 15.76 mm | 31.2 mm | 11.21 mm | 15.68 mm |
| 12228 | | | | |

Table 3 presents the results of the inhibition zones formed in each condition (mm). The antibacterial selectivity can be seen for the B. pseudologum that showed the highest in vitro selectivity, followed by B. longum, relative to S. aureus and S. epidermidis, as the inhibition zones are considerably larger against S. aureus in comparison to S. epidermidis.

### Example 3. Microorganism Removal and Recolonization Inhibition

An evaluation in vivo of the effect of a solid cosmetic composition according to the present invention (soap), compared to a solid composition commercially available (soap) was performed. In this experiment, the manipulation of cell phone, computer, money or any interventions in the hands, or use of any other washing products, etc., was not allowed.

Half of the volunteers washed their hands with the soap according to the present invention and the other half with the soap commercially available (composition that comprises triclosan, triclocarban, DMDM hydantoin, net).

The effectiveness of microorganism removal was higher with the soap according to the present invention than when using the commercially available soap. The soap of the invention cleaned the hands as effectively as antibacterial soap in the art, removing 70% of bacteria and 50% of the fungi. Measurements 30 minutes after the washing showed that the commercially available soap enabled 9 times more bacteria to recolonize and almost 100 times more fungi to recolonize in comparison with the soap of the present invention. Tables 4 and 5 illustrate those results.

TABLE 4
Percentage of microorganisms removed

| % of | | |
|---|---|---|
| microorganisms | present | commercially |
| removed | invention | available |
| bacteria | 69.5 | 79.7 |
| virus | 61.8 | 61.1 |
| fungi | 49.5 | 43.5 |

TABLE 5
Percentage of microorganisms
present on the skin 30 minutes
after the washing

| % of | | |
|---|---|---|
| microorganisms | present | commercially |
| after 30 minutes | invention | available |
| bacteria | 80 | 180 |
| virus | 35 | 125 |
| fungi | −5 | 100 |

The person skilled in the art will promptly evaluate advantages by using the teachings contained in the text and examples herein and will be able to propose variations and equivalent embodiments, without departing from the scope of the invention as defined in the claims.

1. A COSMETIC COMPOSITION comprising a probiotic ingredient selected from Bifidobacteria longum 5^{1A}; Bifidobacteria bifidum 162^{2A}; Bifidobacteria breve 110^{1A}; Bifidobacteria pseudolongum 119^{1A}, or mixtures thereof and cosmetically acceptable excipients.

2. THE COSMETIC COMPOSITION, according to claim 1, comprising the probiotic ingredient in concentration from about 10⁵ to about 10³ cells/ml.

3. USE OF PROBIOTICS AS SELECTIVE ANTIBACTERIAL INGREDIENT TO THE SKIN wherein the probiotics are selected from Bifidobacteria longum 5^{1A}; Bifidobacteria bifidum 162^{2A}; Bifidobacteria breve 110^{1A}; Bifidobacteria pseudolongum 119^{1A}, or a mixture thereof.

4. THE USE, according to claim 5, wherein the probiotic ingredient is in concentrations selected from about 10⁵ to about 10³ cells/ml.

5. USE OF PROBIOTICS TO PREPARE A COSMETIC COMPOSITION FOR CLEANING AND TREATMENT OF THE SKIN, wherein the probiotics are selected from Bifidobacteria longum 5^{1A}; Bifidobacteria bifidum 162^{2A}; Bifidobacteria breve 110^{1A}; Bifidobacteria pseudolongum 119^{1A}, or mixtures thereof.

6. THE USE, according to claim 7, wherein the probiotic ingredient is in concentrations selected from about 10⁵ to about 10³ cells/ml.

7. A METHOD FOR CLEANING AND ANTIAGING TREATMENT OF THE SKIN comprising the application of a cosmetic composition comprising a probiotic ingredient selected from Bifidobacteria longum 5^{1A}; Bifidobacteria bifidum 162^{2A}; Bifidobacteria breve 110^{1A}; Bifidobacteria pseudolongum 119^{1A}, or mixtures thereof and cosmetically acceptable excipients.

8. THE METHOD, according to claim 7, wherein the probiotic ingredient is in concentrations selected from about 10⁵ to about 10³ cells/ml.
